(19) United States
(12) Patent Application Publication
(10) Pub. No.: US 2025/0337338 A1
(43) Pub. Date: Oct. 30, 2025

(54) POWER MODULE HAVING COVER CONTACT ELEMENT AND SINTERED CONNECTION

(71) Applicant: Robert Bosch GmbH, Stuttgart (DE)

(72) Inventors: Erik Sueske, Reutlingen (DE); Andreas Krutsch, Rottenburg (DE); Daniel Feil, Trochtelfingen (DE)

(21) Appl. No.: 18/644,640

(22) Filed: Apr. 24, 2024

(30) Foreign Application Priority Data
Mar. 23, 2023 (DE) 102023202633.3

(51) Int. Cl. H02M 7/00; H01L 23/373; H01L 23/485; H01L 23/498

(52) U.S. Cl. CPC H02M 7/003; H01L 23/3736; H01L 23/485; H01L 23/498; A61B 2562/16; H01L 2224/27505

(57) ABSTRACT

A power module. The power module includes a circuit carrier. A first sintered connecting layer is applied to the circuit carrier. At least one power semiconductor is applied to at least one portion of the first sintered connecting layer. At least one portion of a second sintered connecting layer is applied to the at least one power semiconductor. A portion of an electrical cover contact element is applied to the at least one portion of the second sintered connecting layer.

### FIELD

The present invention relates to a power module, for example for an inverter and/or power switch and/or motor control device, for example for a vehicle, for example an electrically powered vehicle.

### BACKGROUND INFORMATION

Power modules having power semiconductors are used, among other things, in energy converters such as inverters and/or power switches and/or motor control units, for example in electrically powered vehicles.

Typical soft solders generally have a melting temperature in a range of 210° C. to 260° C., although solders with a high lead content can also have a melting temperature of up to approx. 300° C.

During the operation of power modules, however, high temperatures, in particular above 230° C., possibly even briefly above 400° C., can occur, and high thermomechanical loads can occur, which can cause soldered joints to melt and loosen, which can lead to a reduction in the power density and/or service life and/or operational safety of the module.

### SUMMARY

An object of the present invention is to provide a power module, for example for an inverter and/or power switch and/or for a motor control unit, for example for a vehicle, for example an electrically powered vehicle. In particular, the power module has a circuit carrier.

In particular, according to an example embodiment of the present invention, a first sintered connecting layer is applied to the circuit carrier. In particular, at least one power semiconductor is applied to at least one, in particular first, portion of the first sintered connecting layer. In particular, at least one portion of a second sintered connecting layer is applied to the at least one power semiconductor. A portion, in particular a first portion, of an electrical cover contact element is applied to the at least one portion of the second sintered connecting layer.

A sintered connection can be understood in particular as a connection which can be formed or is formed by sintering metal particles, for example silver and/or copper particles, for example at an elevated temperature, in particular below the melting temperature, and for example under increased pressure, for example in a range from 5 MPa to 30 MPa, preferably in a range from 10 or 15 to 25 MPa.

In particular, an electrical cover contact element can be understood to be an electrical contact element designed for the upper-side electrical contacting of power semiconductors, for example, which can be applied or is applied to the upper side of the power semiconductor. Such an electrical cover contact element can also be referred to as a top contact element or top connection, for example. For example, the electrical cover contact element can be designed and/or shaped as a clip.

Since sintered connections have a significantly higher melting temperature than soldered connections, for example silver sintered connections can have a melting point of approximately 962° C. or copper sintered connections can even have a melting point of approximately 1085° C., through the use of a sintered connecting layer, for example instead of a soldered connecting layer, a detachment-resistant connection can be advantageously achieved, even at high temperatures and/or under high thermomechanical loads, both of the at least one power semiconductor to the circuit carrier and in particular also of the electrical cover contact element to the at least one power semiconductor, and in this way the power density and/or the service life and/or the operational safety of the power module can be increased, in particular even at high temperatures and/or under high thermomechanical loads. In addition, sintered connections can advantageously have a comparatively high thermal conductivity, for example on the order of >100 W/mK at room temperature, and can be realized in layer thicknesses of approximately 10 to 100 μm. Therefore, the sintered connecting layer can advantageously be used to realize not only an electrical connection, but also a thermal connection of power semiconductors and, for example, their heat dissipation. This is also an advantageous way of protecting the assembly and connection technology in the event of short-term special operating conditions at high temperatures, for example in the event of a short circuit. Advantageously, costly and/or assembly- and/or connection-restricting measures for limiting the temperature and/or reducing the temperature load, for example in the form of a reduction in resistance by increasing the active semiconductor area, can also be dispensed with.

According to an example embodiment of the present invention, the electrical cover contact element can advantageously be used to electrically connect one power semiconductor or even two or more power semiconductors electrically from above in a simple manner. In addition, the at least one power semiconductor can advantageously also be thermally connected or cooled by the electrical cover contact element. For example, by connecting the electrical cover contact element via the sintered connecting layer, a comparatively high thermal mass can be connected and in this way the at least one power semiconductor can be protected against short-term loads.

Overall, a detachment-resistant electrical and thermal connection of the at least one power semiconductor can thus advantageously be achieved even at high temperatures, for example above 230° C., and/or under high thermomechanical loads, and in this way the power density and/or the service life and/or the operational safety of the power module can be increased, in particular even at high temperatures and/or under high thermomechanical loads, for example in comparison to modules with soldered connections instead of sintered connections.

Such a power module—for example one that is free of casting compound, in particular one that is still free of casting compound—can already be used as such if necessary. In particular, however, such a power module can also be a preliminary stage for a power module with a casting compound, in particular in which the at least one power semiconductor is encapsulated with a casting compound.

In one example embodiment of the present invention, the electrical cover contact element is designed and/or formed as planar. For example, the electrical cover contact element can be designed and/or formed as a contact plate. The power density of the power module can be advantageously increased by a planar connection. For example, the electrical cover contact element can be used to electrically and thermally connect a large part of the surface of the at least one power semiconductor, so that the power semiconductor can advantageously be operated in a homogeneous state or the temperature in the power semiconductor can be homogenized and hot spots can be avoided, for example.

In a further example embodiment of the present invention, the electrical cover contact element is designed for the electrical contacting of two or more power semiconductors, in particular from above or via their upper side. In this way, two or more power semiconductors can advantageously be electrically and, for example, thermally connected simultaneously and/or equally through the electrical cover contact element in a particularly simple and time-saving manner.

In one realization of this embodiment of the present invention, the electrical cover contact element has at least one first portion for the electrical connection, in particular for the electrical and thermal connection, of a power semiconductor. In particular, the electrical cover contact element can have two or more first portions for the electrical connection, in particular for the electrical and thermal connection, of one power semiconductor in each case.

Furthermore, according to an example embodiment of the present invention, the electrical cover contact element can have at least one second portion for the electrical connection, in particular for the electrical and thermal connection, of the cover contact element, in particular itself.

In a particular realization of this embodiment of the present invention, two or more first portions of the electrical cover contact element are each applied to one of two or more portions of the second sintered connecting layer, which are each applied to one of two or more power semiconductors.

In a further example embodiment of the present invention, the electrical cover contact element is a power contact (source connection) or a measuring contact (sensor connection) or a signal contact (signal and/or gate connections).

In a further example embodiment of the present invention, the electrical cover contact element comprises copper and/or silver and/or steel or is made thereof. As already explained in connection with the advantages of sintered connecting layers, copper and silver have high melting points and a high electrical and in particular also thermal conductivity, for which reason the power density and/or the service life and/or the operational safety of the power module can be further increased, in particular at high temperatures and/or under high thermomechanical loads, by forming the electrical cover contact element from copper and/or silver. Steel can advantageously have a CTE adapted to the CTE of SiC and/or Si, so that the thermomechanical load can be reduced by using steel in the electrical cover contact element. If necessary, the electrical cover contact element can be made of coated steel, for example steel coated with copper and/or silver.

In a further example embodiment of the present invention, the at least one second portion of the electrical cover contact element is (also) electrically, in particular electrically and thermally, connected via a sintered connecting layer. For example, the at least one second portion of the electrical cover contact element can be electrically, in particular electrically and thermally, connected via the first sintered connecting layer. For example, the at least one second portion of the electrical cover contact element can be electrically, in particular electrically and thermally, connected via another, in particular second, portion of the first sintered connecting layer. In this way, the power density and/or the service life and/or the operational safety of the power module can advantageously be further increased, in particular at high temperatures and/or under high thermomechanical loads.

In a specific example embodiment of the present invention, the at least one second portion of the electrical cover contact element is formed and/or arranged next to the at least one first portion of the electrical cover contact element and/or to the side of the at least one first portion of the electrical cover contact element and/or the at least one second portion of the electrical cover contact element is electrically, in particular electrically and thermally, connected to the circuit carrier, in particular laterally, for example via a sintered connecting layer, for example via the first sintered connecting layer. In this way, a large thermal capacity can be advantageously connected, which makes it possible to absorb a large amount of heat and, in particular, to dissipate it from the at least one power semiconductor, especially to the side or laterally. In this way, brief thermal loads, for example even of over 400° C., can advantageously be dissipated from the at least one power semiconductor and circuit carrier and in this way the power density and/or the service life and/or the operational safety of the power module can be further increased, in particular also at high temperatures and/or under high thermomechanical loads. In addition, in this way the height of the power module can advantageously be minimized.

In another specific example embodiment of the present invention, however, the at least one second portion of the electrical cover contact element is formed and/or arranged on the at least one first portion of the electrical cover contact element and/or above the at least one first portion of the electrical cover contact element and/or the at least one second portion of the electrical cover contact element is connected, in particular above, electrically, in particular electrically and thermally, to a further component, in particular to a component other than the circuit carrier, for example to an electrical contact, for example to a busbar, or to a housing. In this way, a particularly large heat capacity can be advantageously connected, which makes it possible to absorb a large amount of heat and, in particular, to dissipate it from the at least one power semiconductor and circuit carrier, especially upwards or at the top. In this way, brief thermal loads, for example even of over 400° C., can advantageously be dissipated from the at least one power semiconductor and circuit carrier and in this way the power density and/or the service life and/or the operational safety of the power module, in particular even at high temperatures and/or under high thermomechanical loads, can be still further increased. In addition, the space required on the circuit carrier or the surface area of the circuit carrier can be advantageously reduced, for example minimized. For example, at least one portion of the further component, for example of the electrical contact, for example of the busbar, or of the housing, can be arranged on the at least one second portion of the electrical cover contact element. The portion of the further component arranged on the at least one second portion of the electrical cover contact element, for example the electrical contact, for example the busbar, can be connected to the at least one second portion of the electrical cover contact element, for example by means of a welded connection, i.e., advantageously by means of a direct welded contact. If necessary, a further portion of the further component, for example of the electrical contact, for example of the busbar, or of the housing, can be connected to the circuit carrier, for example to a metal layer of the circuit carrier, for example by means of a further welded connection, i.e., advantageously also by direct welded contact.

In a further example embodiment of the present invention, the first sintered connecting layer and/or the second sintered connecting layer comprises silver and/or copper or is made thereof.

In a further example embodiment of the present invention, the first sintered connecting layer and/or the second sintered connecting layer has an average layer thickness (d₂₀, d₂₁) in a range from 10 μm to 100 μm, preferably in a range from 10 μm to 30 μm. In this way, both a good electrical and a good thermal connection, which can in particular also be thermally and mechanically stable, can advantageously be realized in a cost-efficient and/or space-efficient manner.

In a further example embodiment of the present invention, the at least one power semiconductor has an average height (h₃₀) in a range from 70 μm to 300 μm.

In a further example embodiment of the present invention, the electrical cover contact element has an average material thickness (m₄₀) in a range from 100 μm to 2000 μm. Preferably, the electrical cover contact element has an average material thickness which is, in particular significantly, greater than the average layer thickness (d₂₀) of the first sintered connecting layer and/or is, in particular significantly, greater than the average layer thickness (d₂₁) of the second sintered connecting layer and/or is, in particular significantly, greater than the average height (h₃₀) of the at least one power semiconductor. In this way, the electrical cover contact element can advantageously provide a high thermal capacity and achieve a good heat dissipation capacity, in this way increasing, for example maximizing, the thermal stability of the power module at least in the event of short-term high temperature loads and/or special operating states, for example in the event of an active short circuit. For example, the electrical cover contact element can have an average material thickness that is at least twice, for example at least three times or four times or five times or six times, possibly even at least seven times, as large as the average layer thickness of the first sintered connecting layer and/or the average layer thickness of the second sintered connecting layer. For example, the electrical cover contact element can have an average material thickness that is at least twice as large as the average height of the at least one power semiconductor.

In a further example embodiment of the present invention, the circuit carrier has at least one metal layer. The first sintered connecting layer can be applied in particular to the metal layer of the circuit carrier.

In one realization of this embodiment of the present invention, the metal layer comprises copper or is made thereof. Copper has proven to be advantageous for electrical and electronic applications and can provide a chemical bond for casting compounds through oxide formation. In this way, an increase in the power density and/or service life and/or operational safety of the power module can be advantageously achieved. For example, the metal layer can have an average layer thickness (d₁₁, d_{11′}) in a range from 300 μm to 2000 μm.

In a further example embodiment of the present invention, the circuit carrier also has an insulating layer.

In one realization of this embodiment of the present invention, the insulating layer comprises an inorganic, in particular ceramic, material and/or a plastics material. In particular, the insulating layer can be made of an inorganic, in particular ceramic, material or a plastics material. For example, the insulating layer can comprise a nitridic and/or carbidic and/or oxidic material, for example silicon nitride (Si₃N₄) and/or aluminum nitride (AlN) and/or boron nitride (BN) and/or silicon carbide (SiC) and/or aluminum oxide (Al₂O₃) and/or magnesium oxide (MgO) and/or silicon oxide (SiO₂) and/or beryllium oxide (BeO).

For example, the insulating layer can be made of an inorganic, in particular ceramic, for example nitridic, material, for example silicon nitride (Si₃N₄) and/or aluminum nitride (AlN) and/or boron nitride (BN), or from a plastics material which contains at least one polymer and, for example, furthermore at least one inorganic, in particular ceramic, for example oxidic and/or nitridic and or carbidic filler, for example aluminum oxide (Al₂O₃) and/or magnesium oxide (MgO) and/or silicon oxide (SiO₂) and/or beryllium oxide (Be), and/or silicon nitride (Si₃N₄) and/or aluminum nitride (AlN) and/or boron nitride (BN) and/or silicon carbide (SiC).

The circuit carrier can be a power substrate, for example.

The circuit carrier can, for example, be an AMB (Active Metal Brazing), for example with an inorganic, in particular ceramic, for example nitridic, insulating layer, for example made of silicon nitride (Si₃N₄) and/or aluminum nitride (AlN) and/or boron nitride (BN), and a metal layer soldered thereon. The insulating layer can also be used here as a carrier layer.

The circuit carrier can, for example, also be a DBC (Direct Bond Copper), for example with an inorganic, in particular ceramic, for example nitridic or oxidic, insulating layer, for example made of aluminum nitride (AlN) and/or silicon nitride (Si₃N₄) and/or boron nitride (BN) and/or aluminum oxide (Al₂O₃), and a metal layer, in particular copper or aluminum, applied thereon. The insulating layer can also be used here as a carrier layer if necessary.

However, the circuit carrier can also be an IMS (Insulated Metallic Substrate), for example. In particular, the insulating layer can be made from a plastics material and comprise, for example, at least one polymer and, for example, in addition at least one inorganic, in particular ceramic, for example oxidic and/or nitridic and/or carbidic, filler, for example aluminum oxide (Al₂O₃) and/or magnesium oxide (MgO) and/or silicon oxide (SiO₂) and/or beryllium oxide (BeO), and/or silicon nitride (Si₃N₄) and/or aluminum nitride (AlN) and/or boron nitride (BN) and/or silicon carbide (SiC). The metal layer can be applied to the insulating layer in the form of a foil, for example. The insulating layer can be supported by a carrier layer, for example made of aluminum.

In a further realization of this embodiment of the present invention, the insulating layer has, on at least one side, at least one first portion coated with at least one first portion of the metal layer and a second portion coated with at least one second portion of the metal layer and at least one portion without a metal layer. Through the metal layer-free portions, on the one hand, an electrical and possibly also thermal decoupling of components of the power module can be achieved. On the other hand, due to the fact that the insulating layer has metal layer-free portions and the material of the insulating layer can have better adhesion properties and, in particular, can have a higher proportion of components suitable for the chemical bonding of a casting compound applied thereto, for example in the form of oxidic impurities in nitridic and/or carbidic materials and/or oxides in oxidic materials and/or functional groups in organic, in particular polymeric, materials, than the metal layer, through the metal layer-free portions of the insulating layer an improved adhesion and in particular also a further improved chemical bonding of a casting compound applied to the metal layer-free portions of the insulating layer can be achieved. Advantageously, here epoxy-based and/or other casting compounds, for example those forming hard casting, can form chemical bonds, for example with oxides of the metal layer-free portions of the insulating layer, and in this way a particularly stable and in particular particularly detachment-resistant chemical bonding of a casting compound applied thereto can be achieved. The at least one metal layer-free portion of the insulating layer can for example also be free of sintered connecting layers and/or power semiconductors.

The insulating layer may have a smaller average thickness than the metal layer.

In a particular example embodiment of the present invention, the at least one power semiconductor is applied to one, or the first, portion of the first sintered connecting layer, which is applied to the first portion of the metal layer of the circuit carrier.

In a specific example embodiment thereof, the at least one second portion of the electrical cover contact element can be applied to one or the second portion of the first sintered connecting layer, which is applied to the second portion of the metal layer of the circuit carrier. In this way, the at least one power semiconductor can advantageously be electrically connected to the circuit carrier in a simple and efficient manner by means of the electrical cover contact element and the sintered connecting layers.

In another specific embodiment of the present invention, on the metal layer of the circuit carrier, for example on the first or second portion of the metal layer of the circuit carrier, the further portion of the further component, for example of the electrical contact, for example of the busbar, or of the housing, can be connected to the circuit carrier, in particular to the metal layer of the circuit carrier, for example by means of the further welded connection. In this way, the at least one power semiconductor can advantageously be electrically connected to the circuit carrier in a simple and efficient manner by the electrical cover contact element, the sintered connecting layers and the further component, and at the same time heat can be dissipated away from the at least one power semiconductor and the circuit carrier by the thermal capacity of the electrical cover contact element and the further component.

In a further example embodiment of the present invention, the at least power semiconductor comprises or is a wide-band-gap semiconductor, such as a SiC power semiconductor and/or GaN power semiconductor. Such power semiconductors can advantageously remain stable even at high temperatures and/or under special operating conditions, so that in this way the power density and/or the service life and/or the operational safety can be further increased, in particular even at high temperatures and/or under high thermomechanical loads. With wide-band-gap semiconductors, the functionality—especially with short pulses and/or when briefly exceeding 400° C.—can advantageously still remain.

In a further example embodiment of the present invention, the upper side and/or the bottom side of the at least one power semiconductor is formed by and/or coated with a noble metal layer. In this way, the electrical, in particular electrical and thermal, connection of the at least one power semiconductor can be further improved and in this way the power density and/or service life and/or operational safety of the power module can be further increased.

In a further example embodiment of the present invention, the at least one power semiconductor is encapsulated with a casting compound. Casting with the casting compound can advantageously protect and/or insulate the at least one power semiconductor from environmental influences and/or media. In addition, mechanical stresses can be absorbed by casting with the casting compound, in particular with a hard casting compound. In this way, the service life and/or operational safety of the power module can be further increased. The casting compound can also advantageously increase the power density of the power module, which can have an advantageous effect on the economic implementation of energy converters and/or in the field of electromobility, for example.

In one realization of this embodiment of the present invention, the casting compound is also applied to the at least one metal layer-free portion of the insulating layer. Advantageously, a particularly good adhesion of the casting compound, for example via oxides in these portions, can be achieved and thus a significantly improved service life and/or operational safety of the power module can be achieved and, in particular, the power density of the power module can be further increased, which can have an advantageous effect, for example, on an economical implementation of energy converters and/or in the field of electromobility.

In a further realization of this embodiment of the present invention, the casting compound is a hard casting compound. Hard casting can advantageously absorb mechanical stresses and thus further increase the service life and/or operational safety of the power module. In addition, the power density of the power module can be further increased, which can have an advantageous effect on the economic implementation of energy converters and/or in the field of electromobility, for example. The hard casting compound can be an organic or ceramic hard casting compound, for example.

In a further realization of this embodiment of the present invention, the electrical cover contact element is (also) at least partially encapsulated with the casting compound. In this way, mechanical stresses can also be absorbed by the casting compound, and in this way in turn the service life and/or the operational safety and/or the power density of the power module and thus, for example, the economic implementation of energy converters and/or in the field of electromobility can be further increased.

In a specific example embodiment thereof, the upper side of the electrical cover contact element is also at least partially, if necessary completely, encapsulated with the casting compound. In this way, mechanical stresses can be advantageously absorbed in numerous directions by the casting compound.

In another specific example embodiment thereof, the upper side of the electrical cover contact element is at least partially, in particular completely, free of casting compound. In this way, heat can be better dissipated via the upper side of the electrical cover contact element and, in particular, heat conduction via the casting compound in the direction of the at least one power semiconductor can be reduced, for example minimized.

In particular, the casting compound, in particular the hard casting compound, can be designed to form chemical bonds with oxides. In this way, a chemical bonding of the casting compound, in particular of the hard casting compound, to the circuit carrier, in particular to the metal layer-free portions of the insulating layer, can be advantageously achieved and in this way a particularly stable and in particular particularly detachment-resistant chemical bonding of the casting compound can be achieved.

For example, the casting compound, in particular the hard casting compound, can be organic-based and/or ceramic-based. For example, the casting compound, in particular the hard casting compound, can be epoxy-based or ceramic. In this way, a chemical bonding of the casting compound, in particular the hard casting compound, to the oxides of the layers can be achieved.

In a further example embodiment of the present invention, the circuit carrier has a first metal layer on a first side and a second metal layer on a second side, in particular opposite the first side. In particular, the first metal layer and the second metal layer can be of the same or different design as the metal layer described above.

In a further example embodiment of the present invention, the circuit carrier has an insulating layer, with the first metal layer being applied to a first side of the insulating layer and the second metal layer being applied to a second side of the insulating layer, in particular opposite the first side. The circuit carrier can be an AMB (Active Metal Brazing) or DBC (Direct Bond Copper), for example with an inorganic, in particular ceramic, for example nitridic, insulating layer, for example made of silicon nitride (Si₃N₄) and/or aluminum nitride (AlN) and/or boron nitride (BN) and/or aluminum oxide (Al₂O₃), and a metal layer applied thereon. The insulating layer can also be used here as a carrier layer.

In another example embodiment of the present invention, the circuit carrier has a first insulating layer and a second insulating layer. The first and second insulating layers can be designed and/or formed in the same or different ways as the insulating layer described above. In particular, the first metal layer can be applied to the first insulating layer and the second metal layer to the second insulating layer. The circuit carrier can have a carrier layer, for example made of aluminum, between the first and second insulating layers. The circuit carrier can be an IMS (Insulated Metallic Substrate), for example. In particular, the first and second insulating layers can be formed from a plastics material and, for example, comprise at least one polymer and, for example, furthermore at least one inorganic, in particular ceramic, for example oxidic and/or nitridic and/or carbidic, filler, for example aluminum oxide (Al₂O₃) and/or magnesium oxide (MgO) and/or silicon oxide (SiO₂) and/or beryllium oxide (BeO), and/or silicon nitride (Si₃N₄) and/or aluminum nitride (AlN) and/or boron nitride (BN) and/or silicon carbide (SiC). The metal layers can be applied to the insulating layers in the form of a foil, for example.

With regard to further technical features and advantages of the power module according to the present invention, explicit reference is hereby made to the figure and the description of the figures.

### BRIEF DESCRIPTION OF THE DRAWINGS

Further advantages and advantageous embodiments of the subjects according to the present invention are illustrated by the figures and explained in the following description. It is to be noted that the figures are only descriptive in character and are not intended to limit the present invention in any way.

FIG. 1 shows a schematic cross-section through an example embodiment of a power module according to the present invention having an electrical cover contact element with a lateral second portion.

FIG. 2 shows a schematic cross-section through an example embodiment of a power module according to the present invention having an electrical cover contact element with an upper-side second portion.

FIG. 3 shows a schematic cross-section through an example embodiment of a power module according to the present invention having an electrical cover contact element with an upper-side second portion on which a further component, for example a busbar, is arranged.

### DETAILED DESCRIPTION OF EXAMPLE EMBODIMENTS

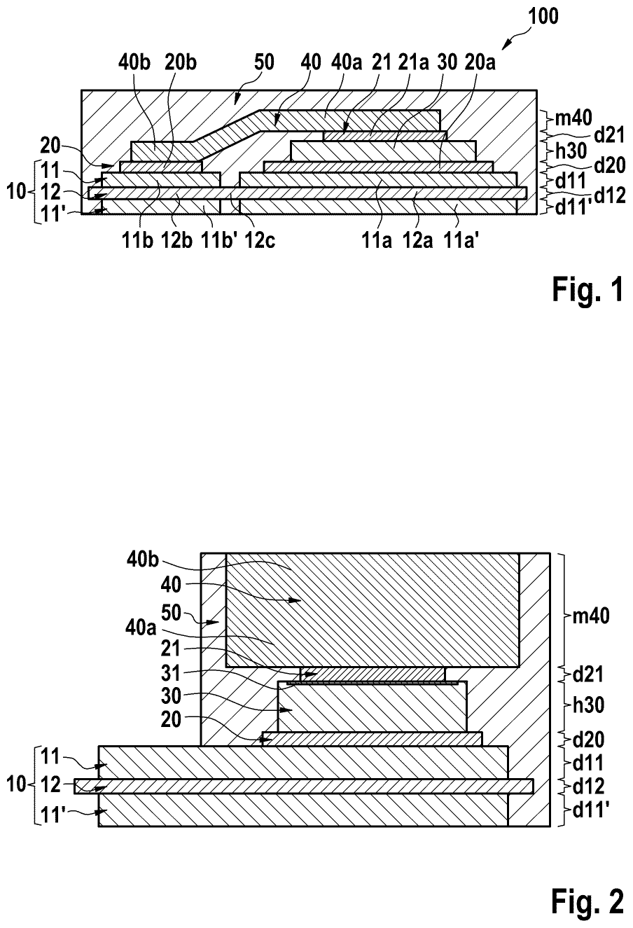

FIG. 1 shows an embodiment of a power module 100 according to the present invention, for example for an inverter and/or power switch, for example for an electrically powered vehicle. Here the power module 100 has one circuit carrier 10.

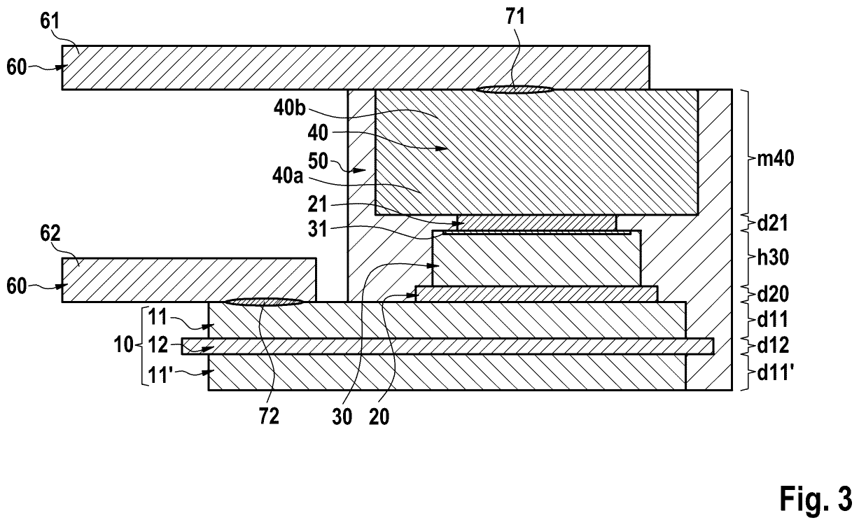

FIG. 1 shows that a first sintered connecting layer 20, for example made of silver and/or copper, is applied to the circuit carrier 10. A power semiconductor 30, for example a wide-band-gap semiconductor such as a SiC or GaN power semiconductor, is applied to a, in particular first, portion 20a of the first sintered connecting layer 20. At least one portion 21a of a second sintered connecting layer 21, for example made of silver and/or copper, is again applied to the power semiconductor 30. The upper side of the power semiconductor 30 can here be formed by and/or coated with a noble metal layer (see 31 in FIGS. 2 and 3) in order to improve the connection of the second sintered connecting layer 21.

FIG. 1 further shows that, in turn, one, in particular first, portion 40a of an electrical cover contact element 40, for example made of copper and/or silver and/or steel, is applied to the at least portion 21a of the second sintered connecting layer 21. The electrical cover contact element 40 is here designed and/or formed in particular as planar. For example, the electrical cover contact element 40 can be designed and/or formed as a contact plate. For example, the electrical cover contact element 40 can be a power contact or a measuring contact or a signal contact.

FIG. 1 illustrates that the electrical cover contact element 40 has a first portion 40a for the electrical connection of a power semiconductor 30 and a second portion 40b for the electrical connection of the cover contact element 40.

In the embodiment shown in FIG. 1, the second portion 40b of the electrical cover contact element 40 is formed and/or arranged next to the first portion 40a of the electrical cover contact element 40 and/or laterally to the first portion 40a of the electrical cover contact element 40. The second portion 40b of the electrical cover contact element 40 is electrically connected laterally to the circuit carrier 10. The second portion 40b of the electrical cover contact element 40 is electrically connected in particular via the first sintered connecting layer 20. In particular, the second portion 40b of the electrical cover contact element 40 is electrically connected via another, in particular second, portion 20b of the first sintered connecting layer 20.

FIG. 1 also shows that the circuit carrier 10 has a first metal layer 11, an insulating layer 12, and a second metal layer 11′. To simplify matters, the first sintered connecting layer 20 in FIG. 1 is only applied to the first metal layer 11 of the circuit carrier 10. In principle, however, the structure shown in FIG. 1 on the first metal layer 11 can also be realized alternatively or additionally, in the same or a different way, on the second metal layer 11′.

FIG. 1 illustrates that the insulating layer 12 has on one side a first portion 12a coated with a first portion 11a of the first metal layer 11, and a second portion 12b coated with a second portion 11b of the first metal layer 11, and a metal layer-free portion 12c. Here the power semiconductor 30 is applied to a first portion 20a of the first sintered connecting layer 20, which is applied to the first portion 11a of the first metal layer 11 of the circuit carrier 10. The second portion 40b of the electrical cover contact element 40 is applied to a second portion 20b of the first sintered connecting layer 20, which is applied to the second portion 11b of the second metal layer 11 of the circuit carrier 10.

FIG. 1 also shows that in this embodiment the power semiconductor 30 and also the electrical cover contact element 40 are encapsulated with a casting compound 50. The casting compound 50 can here be in particular a hard casting compound. FIG. 1 shows that the casting compound 50 is also applied to the metal layer-free portion 12c of the insulating layer 12. FIG. 1 also shows that in this embodiment, the upper side of the electrical cover contact element 40 is also at least partially, in particular completely, encapsulated with the casting compound 50.

Finally, FIG. 1 indicates that the first sintered connecting layer 20 and/or the second sintered connecting layer 21 can have an average layer thickness d₂₀, d₂₁ which can be, in particular significantly, smaller than the average material thickness m₄₀ of the electrical cover contact element 40 and/or than the average layer thickness d₁₁, d_{11′} of the, in particular first and/or second, metal layer 11, 11′ and/or than the average layer thickness d₁₂ of the insulating layer 12 and/or than the average height h₃₀ of the power semiconductor 30.

The embodiment shown in FIG. 2 differs from the embodiment shown in FIG. 1 in that the second portion 40b of the electrical cover contact element 40 is formed and/or arranged on the first portion 40a of the electrical cover contact element 40 and/or above the first portion 40a of the electrical cover contact element 40 and in that the upper side of the electrical cover contact element 40 is at least partially, in particular completely, free of casting compound. The second portion 40b of the electrical cover contact element 40 can be electrically connected, for example at the top or via its upper side, to a further component, for example to a housing (not shown).

The embodiment shown in FIG. 3 differs from the embodiment shown in FIG. 2 in that the further component 60, in particular in the form of a busbar, is shown. FIG. 3 shows that a portion 61 of the further component 60, in particular of the busbar, is arranged on the second portion 40b of the electrical cover contact element 40. The portion 61 of the further component 60, in particular of the busbar, arranged on the second portion 40b of the electrical cover contact element 40 is connected to the second portion 40b of the electrical cover contact element 40 by means of a welded connection 71.

FIG. 3 also shows that a further portion 62 of the further component 60, in particular of the busbar, is connected to the circuit carrier 10, in particular to the metal layer 11 of the circuit carrier 10, by means of a further welded connection 72.

1-15. (canceled)

16. A power module, comprising:
a circuit carrier;
a first sintered connecting layer applied to the circuit carrier;
at least one power semiconductor applied to at least one portion of the first sintered connecting layer;
at least one portion of a second sintered connecting layer applied to the at least one power semiconductor; and
a portion of an electrical cover contact element applied to the at least one portion of the second sintered connecting layer.

17. The power module according to claim 16, wherein the electrical cover contact element is planar, and wherein the electrical cover contact element is a contact plate.

18. The power module according to claim 16, wherein the electrical cover contact element is configured for electrical contacting of two or more power semiconductors.

19. The power module according to claim 16, wherein the electrical cover contact element has at least one first portion for electrical connection of a power semiconductor, and wherein the electrical cover contact element further includes at least one second portion for an electrical connection of the cover contact element.

20. The power module according to claim 19, wherein two or more first portions of the electrical cover contact element are each applied to one of two or more portions of the second sintered connecting layer, which are each applied to one of two or more power semiconductors.

21. The power module according to claim 19, wherein:
(i) the at least one second portion of the electrical cover contact element is arranged next to the at least one first portion of the electrical cover contact element and/or to a side of the at least one first portion of the electrical cover contact element; and/or
(ii) the at least one second portion of the electrical cover contact element is electrically connected laterally to the circuit carrier, the at least one second portion of the electrical cover contact element being electrically connected via the first sintered connecting layer.

22. The power module according to claim 19, wherein:
(i) the at least one second portion of the electrical cover contact element is arranged on the at least one first portion of the electrical cover contact element and/or above the at least one first portion of the electrical cover contact element; and/or
(ii) the at least one second portion of the electrical cover contact element is electrically connected, at a top, to a further component, the further component being a component other than the circuit carrier, the further component being an electrical contact, or a busbar, or a housing, at least one portion of the further component being arranged on the at least one second portion of the electrical cover contact element, wherein the portion of the further component arranged on the at least one second portion of the electrical cover contact element is connected to the at least one second portion of the electrical cover contact element by a welded connection, and wherein a further portion of the further component is connected to a metal layer of the circuit carrier by a further welded connection.

23. The power module according to claim 16, wherein the electrical cover contact element is a power contact or a measuring contact or a signal contact.

24. The power module according to claim 16, wherein the electrical cover contact element is made of coated steel coated with copper and/or silver.

25. The power module according to claim 16, wherein:
(i) the first sintered connecting layer and/or the second sintered connecting layer has an average layer thickness in a range from 10 μm to 100 μm; and/or
(ii) the at least one power semiconductor has an average height in a range from 70 μm to 300 μm; and/or
(iii) the electrical cover contact element has an average material thickness in a range from 100 μm to 2000 μm; and/or
(iv) the electrical cover contact element has an average material thickness which is greater than an average layer thickness of the first sintered connecting layer and/or than an average layer thickness of the second sintered connecting layer and/or is greater than an average height of the at least one power semiconductor.

26. The power module according to claim 16, wherein the circuit carrier includes at least one metal layer, and wherein the first sintered connecting layer is applied to the metal layer of the circuit carrier.

27. The power module according to claim 26, wherein:
(i) the circuit carrier further has an insulating layer, wherein the insulating layer has, on at least one side, at least one first portion coated with at least one first portion of the metal layer and a second portion coated with at least one second portion of the metal layer and at least one portion without a metal layer, wherein the at least one power semiconductor is applied to a first portion of the first sintered connecting layer, which is applied to the first portion of the metal layer of the circuit carrier, and wherein the at least one second portion of the electrical cover contact element is applied to a second portion of the first sintered connecting layer, which is applied to the second portion of the metal layer of the circuit carrier, or
(ii) on the metal layer of the circuit carrier, a further portion of a further component is connected to metal layer of the circuit carrier by a further welded connection, the further component being an electrical contact, or a busbar, or a housing.

28. The power module according to claim 16, wherein an upper side and/or a bottom side of the at least one power semiconductor is formed by and/or coated with a noble metal layer.

29. The power module according to claim 27, wherein the at least one power semiconductor is encapsulated with a casting compound, the casting compound being a hard casting compound, and wherein the casting compound is also applied to the at least one metal layer-free portion of the insulating layer.

30. The power module according to claim 29, wherein the electrical cover contact element is also at least partially encapsulated with the casting compound, and: (i) an upper side of the electrical cover contact element is also at least partially encapsulated with the casting compound, or (ii) the upper side of the electrical cover contact element is at least partially free of the casting compound.
